(19) United States
(12) Patent Application Publication
(10) Pub. No.: US 2008/0033031 A1
(43) Pub. Date: Feb. 7, 2008

(54) ANTIBIOTIC COMPOUNDS, COMPOSITIONS AND METHODS OF TREATMENT

(75) Inventors: Brid DEVLIN, King of Prussia, PA (US); Wendy CHERN, Los Gatos, CA (US); Linda MAHONEY, Phoenixville, PA (US); Pahala SIMAMORA, Chester Springs, PA (US); Vijendra NALAMOTHU, Basking Ridge, NJ (US); Gregory GILMARTIN, Schwenksville, PA (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 11/749,791

(22) Filed: Aug. 13, 2007

Related U.S. Application Data
(63) Continuation of application No. PCT/US05/42045, filed on Nov. 18, 2005.
(60) Provisional application No. 60/629,588, filed on Nov. 19, 2004.

(51) Int. Cl. A61K 31/40; A61P 17/00

(52) U.S. Cl. 514/422

(57) ABSTRACT

Antibiotic compounds and compositions and methods for the treatment of dermatological conditions including a sulfoxide or sulfone derivative of an antibiotic from the lincomycin family of antibiotics, or pharmaceutically acceptable salts thereof, optionally including a peroxide component.

### FIELD OF THE INVENTION

This invention relates to new antibiotic compounds, pharmaceutical compositions and methods useful for the topical treatment of acne, rosacea, and other dermatological conditions, and to the use of antibiotics selected from the sulfoxide or sulfone derivatives of compounds of the lincomycin family.

### BACKGROUND OF THE INVENTION

Acne is an inflammatory disease which is very common at puberty and which occurs in skin areas where sebaceous glands are largest, most numerous, and most active. In its milder forms, acne is a superficial disorder which is evidenced by slight, spotty irritations, and which can be treated satisfactorily by ordinary skin hygiene. However, pilosebaceous follicles occur and result in the formation of pustules, infected cysts and, in extreme cases, canalizing inflamed and infected sacs, which may become extensive and leave permanent, disfiguring scars. One aspect of the present invention relates to an improved topical anti-acne composition.

Exemplary antibiotics incorporated in compositions are disclosed in U.S. Pat. No. 3,969,516 (lincomycin family), GB Pat. Specification No. 1,594,314 (erythromycin); and U.S. Pat. No. 3,952,099 (tetracycline). Compositions containing a peroxide are reported in U.S. Pat. Nos. 3,535,422; 4,056,611; 4,387,107. Combinations of erythromycin and peroxides are reported in GB Pat. Specification No. 1,594,314 and U.S. Pat. No. 4,497,794. Antibiotic-containing compositions which also include anti-inflammatory steroids are disclosed in U.S. Pat. No. 4,132,781.

Topical anti-acne preparations which include combinations of a peroxide, such as benzoyl peroxide and an antibiotic, such as an antibiotic of the lincomycin family, are disclosed in U.S. Pat. Nos. 5,466,028, 5,767,098 and 6,013,637, all of which are incorporated herein by reference. U.S. Pat. No. 5,690,923 relates to compositions containing retinoids and clindamycin and its derivatives. However, it has been found that in compositions comprising benzoyl peroxide and antibiotics of the lincomycin family, interaction of the peroxide with the lincomycin antibiotic can result in degradation of the antibiotic. There is, therefore, a need for an antibiotic which has greater stability in formulations with peroxides.

### SUMMARY OF THE INVENTION

In accordance with the present invention, it has been found that sulfoxide and sulfone (hereinafter collectively “SOx”) derivatives of antibiotics from the lincomycin family have greater stability in formulations containing peroxides other than lincomycin antibiotics previously used in such formulation. Thus, the present invention provides a composition for the treatment of acne, rosacea and other dermatological conditions comprising a topically effective amount of an SOx derivative of an antibiotic of the lincomycin family. One embodiment of the present invention includes the SOx derivatives of clindamycin, hereinafter referred to collectively as CSOx.

The formulation may further comprise a topically effective amount of a peroxide. Another aspect of the present invention is to provide a stable composition comprising a topically effective combination of an SOx derivative of an antibiotic of the lincomycin family and a peroxide. Benzoyl peroxide (BPO) is a particularly suitable peroxide for use in the present invention.

Another aspect of the present invention is to provide a method for the treatment of acne, rosacea or other dermatological conditions comprising the topical administration, to a patient afflicted therewith, of a topically effective amount of an SOx derivative of an antibiotic of the lincomycin family, optionally in combination with a topically effective peroxide.

These and other aspects of the present invention are described in more detail below.

### DESCRIPTION OF THE DRAWINGS

FIG. 1A is a graph showing the stability of 1% clindamycin analogs with 5% BPO in pH 3 buffer solution at room temperature (RT), i.e., ambient temperature within the range of 15 to 30° C.

FIG. 1B is a graph showing the stability of 1% clindamycin analogs with 5% BPO in pH 3 buffer solution at 50° C.

FIG. 2A is a graph showing stability of 1% clindamycin analogs with 5% BPO in pH 5 buffer solution at RT.

FIG. 2B is a graph showing stability of 1% clindamycin analogs with 5% BPO in pH 5 buffer solution at 50° C.

FIG. 3A is a graph showing stability of 1% clindamycin analogs with 5% BPO in pH 6 buffer solution at RT.

FIG. 3B is a graph showing stability of 1% clindamycin analogs with 5% BPO in pH 6 buffer solution at 50° C.

FIG. 4 is a graph showing the pH of clindamycin sulfoxide A and BPO samples at RT and 50° C.

FIG. 5 is a graph showing the pH of clindamycin sulfoxide B and BPO samples at RT and 50° C.

FIG. 6 is a graph showing the pH of clindamycin sulfone and BPO samples at RT and 50° C.

FIG. 7 is a graph showing the pH of clindamycin phosphate and BPO samples at RT and 50° C.

FIG. 8 is a graph showing the pH of clindamycin HCl and BPO samples at RT and 50° C.

FIG. 9 is a scaled chromatogram of clindamycin sulfoxide A.

FIG. 10 is a scaled chromatogram of clindamycin sulfoxide B.

FIG. 11 is a scaled chromatogram of clindamycin sulfone.

In the drawings, the following abbreviations are used:
CSOA—clindamycin sulfoxide A isomer
CSOB—clindamycin sulfoxide B isomer
CSO2—clindamycin sulfone
ClinPO4—clindamycin phosphate
ClinHCl—clindamycin hydrochloride.

### DETAILED DESCRIPTION

The term “antibiotic of the lincomycin family” is used herein to refer to a class of antibiotic substances originally recovered from streptomyces lincolnensis.

Exemplary antibiotics of this type include lincomycin and clindamycin and their pharmaceutically acceptable salts and esters such as hydrochlorides and phosphates. Lincomycin is a derivative of the amino acid trans-L-4-α-propyl-hygrinic acid coupled to an octose derivative comprising a pyran ring substituted by a methylsulfanyl group, and may be designated as (2S-trans)-methyl 6,8-dideoxy-6-[[(1-methyl-4-propyl-2-pyrrolidinyl)-carbonyl]-amino]-1-thio-D-erythro-α-D-galacto-octopyranoside.

Preferred antibiotics of lincomycin family for use in the present invention may be represented by the following formula I: 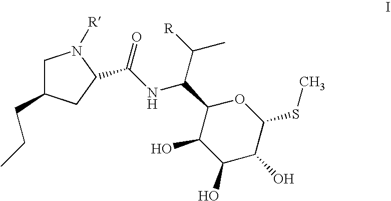
wherein R is hydroxy or halogen, and R′ is hydrogen or methyl, and pharmaceutically acceptable salts and esters thereof.

When R is Cl and R′ is methyl, the compound is clindamycin. When R is Cl and R′ is hydrogen, the compound is N-demethyl clindamycin. When R is OH and R′ is methyl, the compound is lincomycin. When R is OH and R′ is hydrogen, the compound is N-demethyl lincomycin.

The term “sulfoxide or sulfone derivative of an antibiotic of the lincomycin family” means a lincomycin antibiotic in which the sulfanyl linkage of the methylsulfanyl group of the pyran ring has been replaced by a sulfoxide (SO) or sulfone (SO₂) linkage, collectively referred to hereinafter as an “SOx” linkage.

Preferred sulfoxide derivatives of antibiotics of the lincomycin family may be represented by the following formula I(a):

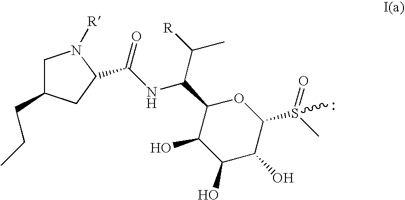

wherein R and R′ are as defined above. The sulfur atom of the sulfoxide group is a chiral site by which two stereoisomeric forms of the sulfoxide may form.

Preferred sulfone derivatives of antibiotics of the lincomycin family may be represented by the following formula I(b): 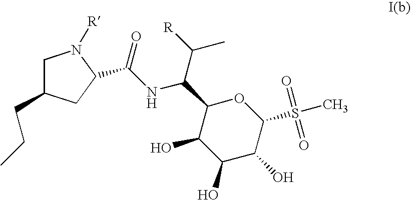
wherein R and R′ are as defined above.

Clindamycin is the 7-deoxy, 7-chloro derivative of lincomycin, and may be designated as (2S-trans)-methyl7-chloro-6,7,8-trideoxy-6-[[(1-methyl-4-propyl-2-pyrrolidinyl)-carbonyl]-amino]-1-thio-L-threo-α-D-galacto-octopyranoside, or as 1-methyl-4-propyl-pyrrolidine-2-carboxylic acid [2-chloro-1-(3,4,5-trihydroxy-6-methylsulfanyl-tetrahydro-pyran-2-yl)-propyl]-amide. Clindamycin may be represented by the following formula II:

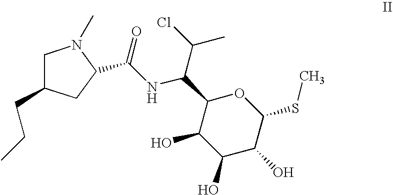

The lincomycin antibiotics are described in U.S. Pat. Nos. 3,475,407; 3,509,127; 3,544,551 and 3,513,155.

In one embodiment of the present invention the lincomycin SOx derivative is clindamycin sulfoxide of the following formula III:

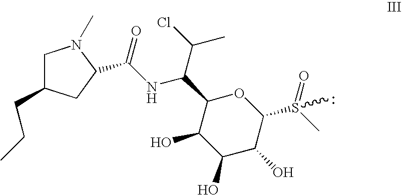

As noted above, the sulfur atom of the sulfoxide group is a chiral site by which two stereoisomeric forms of the lincomycin sulfoxide derivative, in this case clindamycin sulfoxide, may form. The clindamycin sulfoxide compound of formula III may include one or both stereoisomeric forms of clindamycin sulfoxide selected from the following formulas III(a) or III(b):

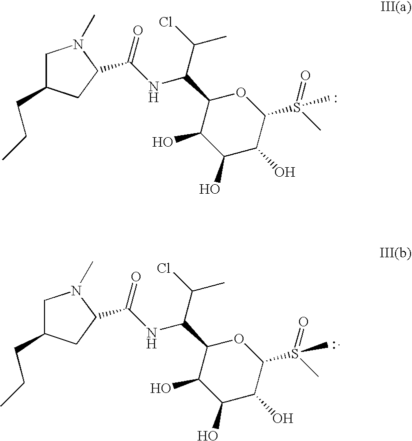

The sulfoxide derivatives of either the antibiotics of the lincomycin family in accordance with the present invention also include the phosphate sulfoxide derivatives, such as the 2-phosphate sulfoxide derivatives of the following formula IV: 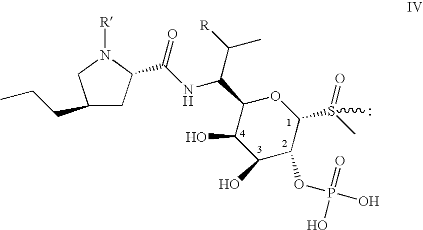
wherein R and R′ are as defined above. The phosphate sulfoxide derivative can also be the 3-phosphate or 4-phosphate derivative.

In a particular embodiment of the present invention, the SOx derivative of the antibiotic of the lincomycin family is clindamycin-2-phosphate sulfoxide, which may be represented by the following formula V:

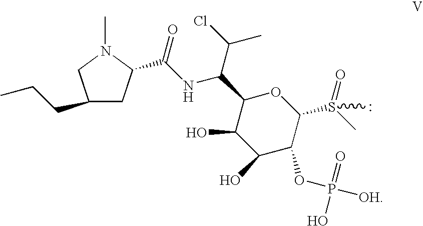

The clindamycin-2-phosphate sulfoxide compound of formula V may include one or both stereoisomeric forms of clindamycin-2-phosphate sulfoxide selected from the following formulas V(a) or V(b):

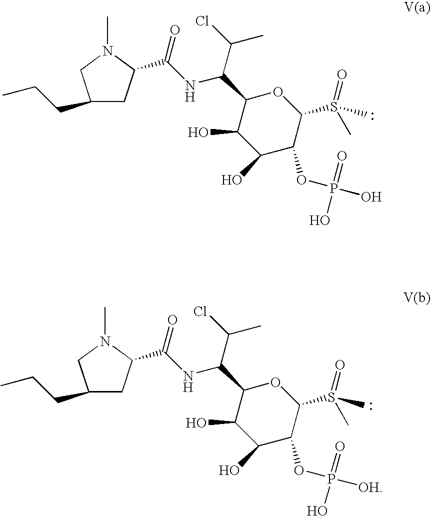

In another embodiment of the invention, the lincomycin SOx derivative is the sulfone derivative, and may be represented by the following formula VI: 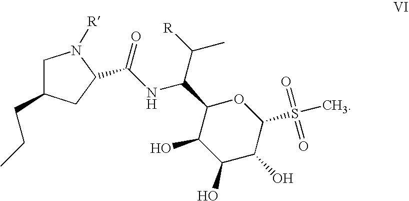
where R is hydroxy or halogen, and R′ is hydrogen or methyl, and pharmaceutically acceptable salts and esters thereof.

A particular lincomycin sulfone for use in the present invention is clindamycin sulfone, which may be represented by the following formula VI(a):

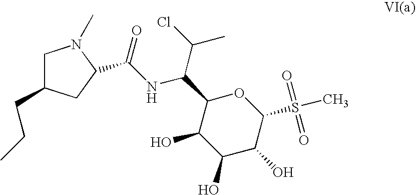

In a particular embodiment of the present invention, the lincomycin SOx derivative is administered in combination with a peroxide. The term “peroxide” means an organic compound containing an oxygen-oxygen bond capable of cleaving and forming oxygen free-radicals. The peroxides include peroxyacids of carboxylic acids, peroxyesters of carboxylic acids and the dimeric product of carboxylic peroxyacids. Exemplary peroxides include t-butyl peroxyesters of straight and branched chain aliphatic carboxylic acids, and dimeric peroxides such as lauroyl peroxide and benzoyl peroxide. A particular peroxide suitable for use in the present invention is benzoyl peroxide, more particularly micronized benzoyl peroxide.

The method of the present invention comprises the administration of an antibiotic selected from the SOx derivatives of an antibiotic of the lincomycin family, or a pharmaceutically acceptable salt thereof. The SOx derivative antibiotic may be administered alone or in combination with other active ingredients. In one embodiment, the SOx derivative antibiotic may be administered to the skin of a patient suffering from acne either simultaneously with or shortly prior to or after the application of a peroxide, as defined above. Accordingly, the two ingredients may be applied to the skin as a mixture or they may separately be applied to the skin. In the latter practice the antibiotic is applied first to the skin and immediately or shortly thereafter the peroxide in applied. Or, the order of application may be reversed.

In an embodiment of the present invention, the composition includes benzoyl peroxide and an SOx derivative of a lincomycin family antibiotic. In one particular form, the composition comprising micronized benzoyl peroxide and an SOx derivative of clindamycin or a pharmaceutically acceptable salt or ester thereof. Examples of pharmaceutically acceptable salts are formed by the combination of the lincomycin family antibiotic with hydrochloric acid, nitric acid, succinic acid, maleic acid, fumaric acid, tartaric acid, etc. Pharmaceutically acceptable esters include phosphate, succinate, etc., particularly at the 2-position in the sugar moiety.

Particularly good results are obtained using the clindamycin sulfoxide of formula III, including one or both of the chiral forms of clindamycin sulfoxide represented by formulas III(a) and III(b), and using the clindamycin sulfone of formula VI(a).

In a particular embodiment, the antibiotic should be present in the composition in an amount of about 0.01 to about 10 weight percent of the total composition, and more particularly from about 0.1 to about 5 weight percent.

When the composition includes a peroxide component, the peroxide is present in the composition in an amount of about 0.1% to about 30%, and particularly about 2.5% to about 15%, all percentages herein by weight and based on the total weight of the composition, unless noted otherwise. More particularly, the amount of peroxide may be about 3% to about 10%. Desirably, the peroxide should be of high purity. An exemplary material includes peroxide in an amount which is not less than about 98% of the stated concentration on the labeled raw material and in the form of finely divided crystalline particles, such as micronized particles having a mean average particle size of less than about 35 microns.

One composition is in the form of an aqueous gel, and which may include an aqueous alcoholic gel. However, liquid suspensions and emulsions, as well as creams, solutions, foams, pastes, lacquers, ointments and powders are acceptable.

The gelling agent used in the aqueous gel composition of this invention may be selected both as to type and quantity to give products of various viscosities. A variety of gelling agents, either naturally occurring or synthetic, may be used for the present purposes. Suitable gelling agents include, but not limited to, cellulose based polymers such as methyl cellulose, ethyl cellulose, hydroxyethyl cellulose, hydroxypropyl methyl cellulose, carboxymethyl cellulose; polyacrylamide; an acrylamide/sodium acryloyldimethyl taurate copolymer product (such as Simulgel®); aluminum magnesium silicate (such as Veegum®)); guar gum; carrageenan; acacia; xanthan gum; zein; polyethylene-propylene glycol copolymers, such as poloxamers; polyoxyethylene alkyl ethers (such as Brij®); acrylate/alkyl acrylate crosspolymers such as Pemulen® and polyacrylic acids, such as carbomers.

The amount of gelling agent included in the present gel composition can range from about 0.1 to about 20% by weight, and particularly from about 0.5 to about 5% by weight, based on the total weight of the composition.

The composition of the present invention may include a surface active agent or dispersing agent to disperse uniformly the active ingredients. A composition may also include a second surface active agent. Such agents include the esters of polyols and sugars, the products of the condensation of ethylene oxide with fatty acids, fatty alcohols, long-chain alkylphenols, long-chain mercaptans, long chain amides, polyethers of polyhydroxylated fatty alcohols and alkylpolyglycol ethers which are included in an amount of from about 0.1% to about 6% by weight.

Another embodiment of the composition of the present invention has a pH which is effective in stabilizing the antibiotic and peroxide ingredients over time. An effective stabilizing pH is in the range of about 2 to about 7, particularly about 3 to about 5, more particularly about 4.

A further composition of the present invention may include a stabilizing agent which acts as an effective barrier to the possible degradative interaction of the peroxide and the antibiotic and/or antioxidant(s). A suitable stabilizing concentration is about 0.1 to about 6% by weight, and more particularly about 0.5% to about 3% by weight. Suitable antioxidants include those commonly used in the medicinal topical preparations, such as alpha-tocopherol, ascorbic acid, etc.

One type of preparation comprises a composition in which the two active ingredients are pre-mixed (as in one component system) and stable at temperatures commonly employed for the storage of clindamycin solutions. Another type of preparation may comprise a two-component system, wherein one component comprises the antibiotic and the other component comprises the peroxide component. Conventional pharmaceutical processes may be used in making up these common forms of medicinal, topical compositions.

As mentioned above, a basic type of topical preparation comprises a mixture of powdered peroxide and antibiotic with an inert diluent. Such a preparation should be sparingly applied to the skin.

The following examples are illustrative of the present invention.

### EXAMPLE 1

The following ingredients may be mixed together to form a powder which may be dusted on the affected skin area, from one to four times a day.

| | W/W Percent |
|---|---|
| Benzoyl peroxide | 0.1-30 |
| Calcium phosphate | 63-98.5 |
| CSOx | 0.1-5 |

### EXAMPLE 2

A liquid suspension of the present invention may be prepared by combining the following ingredients.

| | W/W Percent |
|---|---|
| CSOx | 0.1-5 |
| Benzoyl peroxide | 0.1-30 |
| Surfactant | 0.1-5 |
| Water, purified | Q.S.-100 |

Other preparations which are representative of the present invention, include the following examples.

### EXAMPLE 3

A cream may be prepared by mixing the following ingredients in a suitable container.

| | W/W Percent |
|---|---|
| Polyoxyl 40 Stearate | 4 |
| Stearic Acid | 3 |
| Cetyl Alcohol | 4 |
| Sorbitan Monostearate | 1 |
| Isopropyl Myristate | 1.8 |
| CSOx | 0.1-5 |
| Benzoyl peroxide | 0.1-30 |
| Glycerin | 8 |
| Xanthan Gum | 0.05 |
| Water, purified | QS-100 |

### EXAMPLE 4

A gel according to the present invention is prepared by combining the following ingredients.

| | W/W Percent |
|---|---|
| CSOx | 0.1-5 |
| Benzoyl peroxide | 0.1-30 |
| Carbomer | 2 |
| Disodium lauryl sulfosuccinate | 0.04 |
| Sodium hydroxide | 0.3 |
| Edetate disodium | 0.1 |
| Glycerin | 4 |
| Silicon dioxide | 0.25 |
| Dimethicone | 0.1 |
| Poloxamer 182 | 0.2 |
| Water, purified | Q.S.-100 |

### EXAMPLE 5

A gel according to the present invention is prepared by combining the following ingredients.

| | W/W Percent |
|---|---|
| CSOx | 0.1-5 |
| Veegum ® | 1.5 |
| Carbomer | 1 |
| Dioctyl sodium sulfosuccinate | 1 |
| Diisopropanolamine | 0.75 |
| Ethyl alcohol | 35 |
| Benzoyl peroxide | 0.1-30 |
| Water, purified | Q.S.-100 |

### EXAMPLE 6

A two-part suspension is prepared from the following ingredients.

| | W/W Percent |
|---|---|
| First Container | |
| Veegum ® | 1.5 |
| Carbomer | 0.25 |
| Dioctyl sodium sulfosuccinate | 1 |
| Diisopropanolamine | 0.18 |
| Ethyl alcohol | 25 |
| Butylated hydroxyanisole | 0.1 |
| Benzoyl peroxide | 0.1-30 |
| Water, purified | QS-100 |
| Second Container | |
| CSOx | 0.1-5 |

(w/w based on the total composition)

### EXAMPLE 7

An aerosol spray according to the present invention may be prepared as follows. The following ingredients, in amounts within the below indicated ranges, are blended together and the resulting mixture charged into one chamber of a dual chamber aerosol container.

| | W/W percent |
|---|---|
| Benzoyl peroxide | 0.1-30 |
| Calcium phosphate | 65-97 |
| Calcium stearate | 1-10 |
| PPG-15 stearyl ether | 0.5-5 |

Clindamycin sulfoxide or sulfone (0.1-5 wt %) is charged into the second chamber of the container and the container is pressurized with aerosol propellant.

### EXAMPLE 8

An aqueous gel composition may be prepared according to the following formulation:

| | W/W Percent |
|---|---|
| CSOx | 0.1-5 |
| Carbomer | 2 |
| Dioctyl sodium sulfosuccinate | 0.2 |
| Sodium hydroxide | 0.4 |
| Benzoyl peroxide | 0.1-30 |
| Water, purified | QS-100 |

### EXAMPLE 9

An aqueous gel composition may be prepared according to the following formulation:

| | W/W Percent |
|---|---|
| CSOx | 0.1-5 |
| Carbomer | 1.5 |
| Xanthan gum | 1.0 |
| Sodium hydroxide | 0.4 |
| Glycerin | 5.0 |
| Benzoyl peroxide | 0.1-5.0 |
| Water, purified | QS-100 |

### EXAMPLE 10

An aqueous gel composition may be prepared according to the following formulation:

| | W/W Percent |
|---|---|
| CSOx | 0.1-5 |
| Simugel ® | 5.0 |
| Hydroxyethylcellulose | 0.5 |
| Citric acid | 0.03 |
| Benzoyl peroxide | 0.1-5.0 |
| Water, purified | QS-100 |

### EXAMPLE 11

An aqueous gel composition may be prepared according to the following formulation:

| | W/W Percent |
|---|---|
| CSOx | 0.1-5 |
| Simugel ® | 5.0 |
| Xanthan gum | 0.5-1.0 |
| Citric acid | 0.03 |
| Benzoyl peroxide | 0.1-5.0 |
| Water, purified | QS-100 |

Comparative studies have shown unexpectedly that pH is a significant factor in determining the stability of the composition of the present invention. The active ingredients included in the compositions having a pH within the range described above are physically and chemically more stable than the ingredients included in compositions having a pH outside the defined range. This work is discussed in more detail below.

Applicant has found that when compositions having different pH are subjected to accelerated decomposition conditions of 50° C., compositions having a pH below about 3 and above about 7 show signs of clindamycin degradation. However, after 4 weeks, compositions having an initial pH of about 3 to about 5 shows better stability. The degradation of clindamycin sulfoxide or sulfone appears to be negligible as measured by HPLC. The 4-week aged compositions show that at least about 90% of the clindamycin sulfoxide or sulfone is retained in the composition.

The composition of the present invention may be applied to the afflicted skin of an acne sufferer for a period of time on a regular basis such that the acne condition is brought under control. One regimen of treatment comprises the application of the composition from about one to about four times a day.

As discussed above, in the clindamycin sulfoxide (CSO) represented in formula III there is a chiral site at the sulfoxide linkage which allows for two stereoisomeric forms of clindamycin, represented by formulas III(a) and III(b). The stereospecific forms of CSO were separated, such as by HPLC techniques, and are herein designated “CSO A” and “CSO B”. However, it is not presently known which of formulas III(a) or III(b) specifically represents CSO A or CSO B. Stereospecific synthesis processes have also been developed which favor the production of CSO A or CSO B, thus reducing or eliminating the need for a separation step. These synthesis processes are discussed further below.

In the following examples, tests were conducted using stereospecific forms of CSO A and CSO B, as well as various pharmaceutically acceptable salts thereof. Tests were also conducted using clindamycin sulfone (“CSO2”), as represented in formula VI(a). The syntheses for these compounds are discussed further below.

### EXAMPLE 12

### In Vitro Microbiological Evaluation

The microbiological activity of clindamycin analogs was evaluated by measuring the minimum inhibitory concentrations (MIC) of each compound against Staphylococcus aureus (S. aureus) and Propionibacterium acnes (P. acnes). These two microorganisms were selected, as they are commonly known to be the contributing factors for acne. The compounds evaluated were the following:
a. Clindamycin sulfoxide A isomer (CSO A) and its salts
b. Clindamycin sulfoxide B isomer (CSO B) and its salts
c. Clindamycin sulfone (CSO2) and its salts
d. Clindamycin phosphate
e. Clindamycin HCl
f. Erythromycin

Clindamycin phosphate, clindamycin HCl and erythromycin are known commercially available anti-acne compounds and were included in the experiments for comparison purposes (as controls). The MIC is defined as the concentration of the test compound at which growth is completely inhibited. Thus, the lower the MIC value the more potent the compound. The MIC values from four sets of tests are set forth in Tables 1 and 2.

The MIC data in Table 1 shows that the CSO B isomer is more active than both the CSO A and clindamycin phosphate, and that the CSO A is more active than clindamycin phosphate. Clindamycin HCl and erythromycin show the highest potency from all compounds tested.

TABLE 1
MIC Results against S. aureus ATCC
6538 and P. acnes ATCC 1127

MIC (μg/mL)

| Test compound | S. aureus | P. acnes |
|---|---|---|
| CSO B | 1.6 | 3 |
| CSO A | 10 | 25 |
| Clindamycin phosphate USP | 200 | 200 |
| Clindamycin HCl | <0.8 | <0.8 |
| Erythromycin USP | <0.8 | 1.6 |

The data from additional tests presented in Table 2 is consistent with the MIC data presented in Table 1.

TABLE 2
MIC results against S. aureus ATCC
29213 and P. acnes ATCC 11827

| | | MIC (μg/mL) | |
|---|---|---|---|
| Compound | Assay | S. aureus | P. acnes |
| Clindamycin HCl USP | 853 μg/mg | 0.125-0.062 | 0.062 |
| Clindamycin | 879 μg/mg | 16-32 | 2-4 |
| phosphate USP | | | |
| Erythromycin USP | 92.7% | 0.25-0.5 | 0.062 |
| CSO A | 93.6-94.6% | 8-16 | 4-8 |
| CSO A HCl | 96.0% | 16 | 8 |
| CSO A fumarate | 97.7% | 16 | 8 |
| CSO A succinate | 93.8% | 16 | 16 |
| CSO A maleate | 98.3% | 16 | 16 |
| CSO B | 76.2-97% | 1-2 | 0.25-1 |
| CSO B benzoate | 91.2% | 2 | 0.5 |
| CSO B citrate | 91.7% | 2 | 0.5 |
| CSO B fumarate | 80.7% | 2 | 0.5 |
| CSO B HCl | 90.7-97.3% | 2 | 0.5 |
| CSO2 | 60-99.8% | 1-2 | 0.5-1 |
| CSO2 HCl | 94.9-97.0% | 1 | 0.5 |
| CSO2 sulfate | 97.0% | 1 | 0.5 |

### EXAMPLE 13

### Stability Studies with Benzoyl Peroxide

The stability of CSO A, CSO B, CSO2 in buffer solutions of different pHs in the presence of benzoyl peroxide (BPO) was carried out at ambient room temperature (RT) and 50° C. For comparison purposes, clindamycin phosphate and clindamycin HCl were included in the experiment as controls. The concentrations of each of the clindamycin analogs and BPO in the mixtures were 1% (w/w) and 5% (w/w), respectively.

Samples were prepared by dissolving each of the clindamycin analogs and suspending the BPO powder in the buffer solutions of different pHs. Samples were placed either at RT or at 50° C. Samples were pulled at time intervals and tested for the appropriate clindamycin analog and pH.

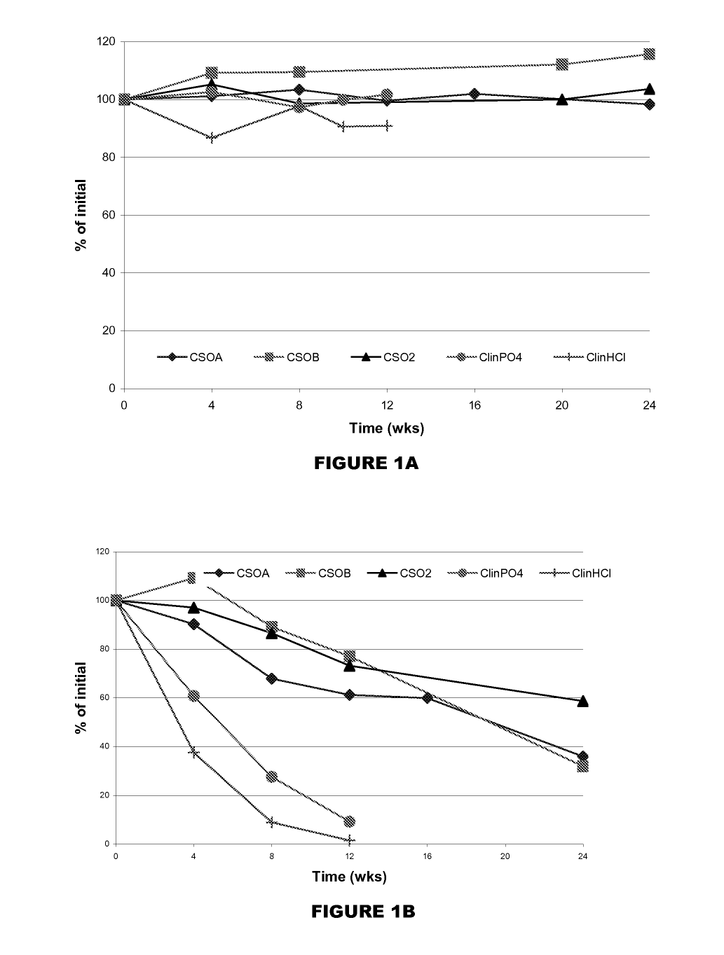

FIGS. 1(A,B), 2(A,B) and 3(A,B) show the stability results of 1% clindamycin analogs in the presence of 5% BPO in buffer solutions pH 3, 5, 6, respectively, at RT and 50° C. The figures show the plots of the percent remaining (% of initial) of the clindamycin analog versus time. The percent of initial is obtained by normalizing the concentration at each time point to the initial concentration.

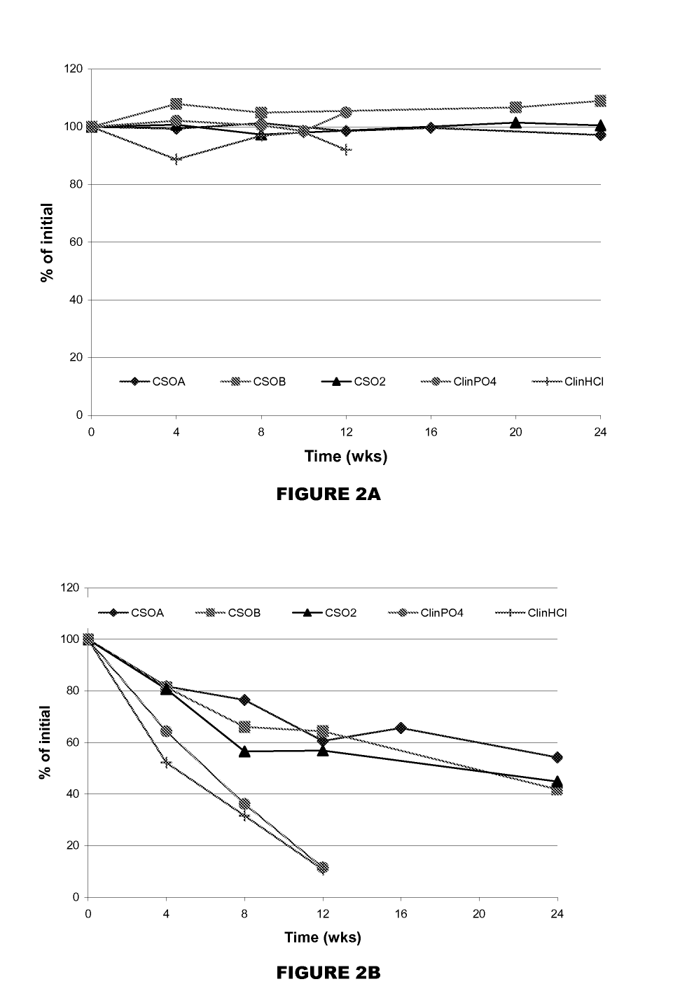

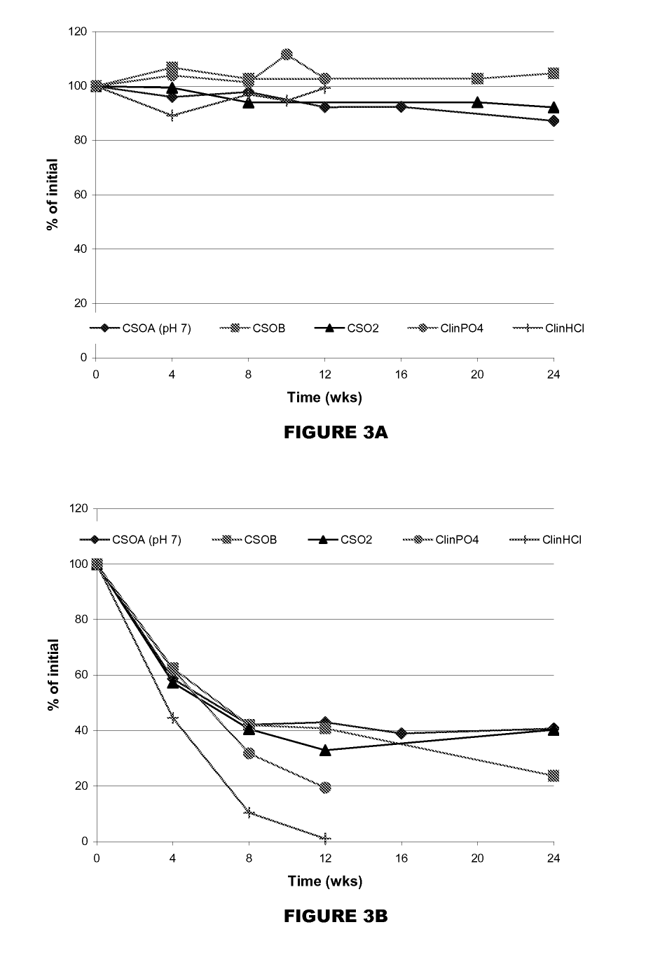

All samples stored at RT showed negligible loss of the active at 12-week time point (24-week time point for the CSOx) (see FIGS. 1A, 2A, and 3A). The decrease in concentration generally was less than 10%. On the other hand, some samples stored at 50° C. showed substantial degradation (see FIGS. 1B, 2B and 3B). The degradation, however, was slower for the CSO A, CSO B and CSO2 compared to the clindamycin phosphate and clindamycin HCl. In general, the higher the pH of the buffer solution and the higher the temperature are, the faster the degradation of the compound.

Clindamycin sulfoxides A and B and clindamycin sulfone prepared in pH 3 buffer at 50° C. were found to be more stable than the clindamycin phosphate and clindamycin HCl. They showed a much slower degradation than both clindamycin phosphate and clindamycin HCl (see FIG. 1B). As early as after 4 weeks of storage, clindamycin phosphate and clindamycin HCl showed 40% and 60% loss of active in the sample, respectively, while the CSO A, CSO B and CSO2 showed less than 10% loss.

After 8 weeks of storage at 50° C., only about 30% of the clindamycin phosphate and less than about 10% of the clindamycin HCl remained in the solution. In comparison, after the same period about 90% of the CSO B, about 85% of the CSO2, and about 70% of the CSO A remained. After 12 weeks of storage, there was about 80% of the CSO B, about 70% of the CSO2 and about 60% of the CSO A remaining in the samples (see FIG. 1B). After 24 weeks of storage, the CSOx concentrations remaining in the samples were about 40-60%.

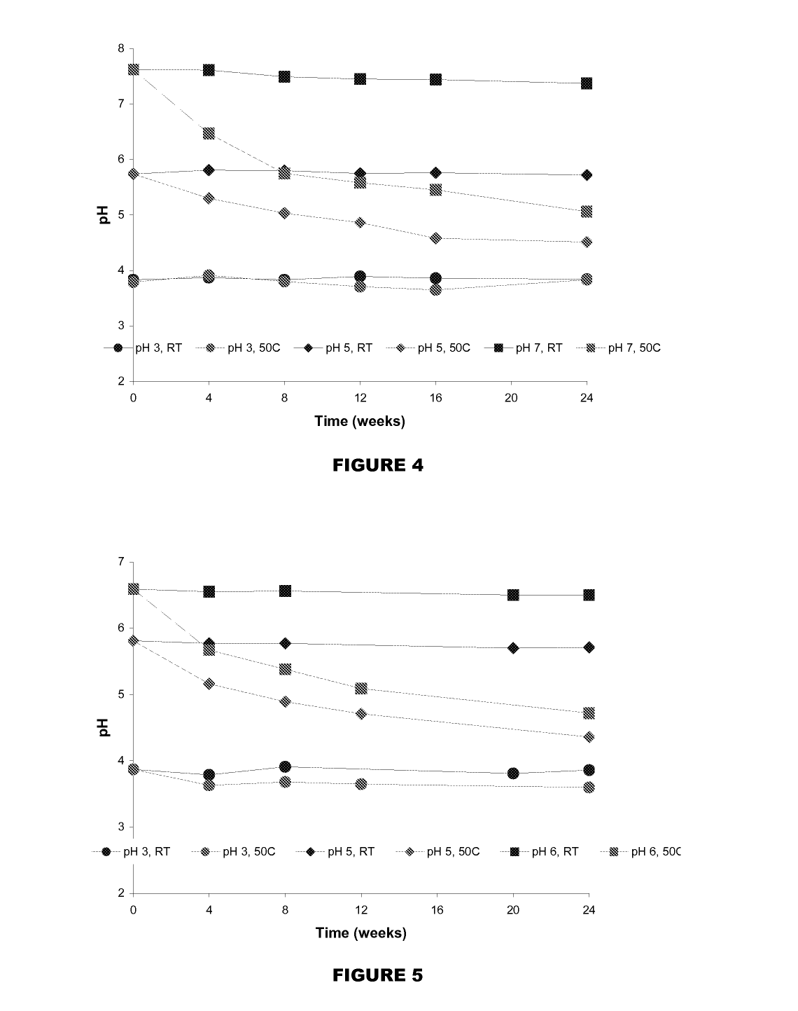

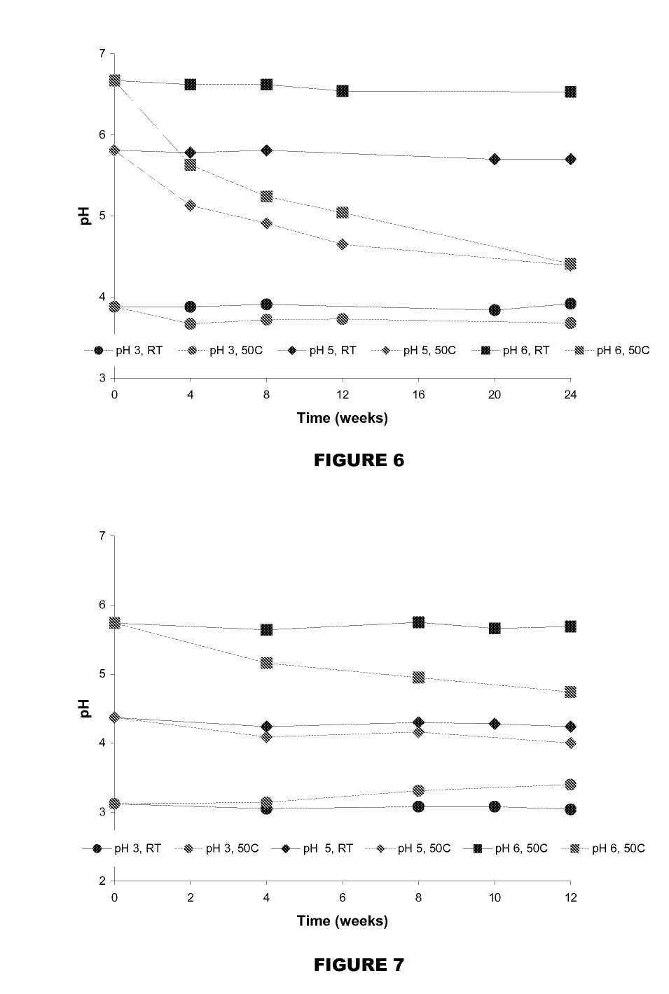

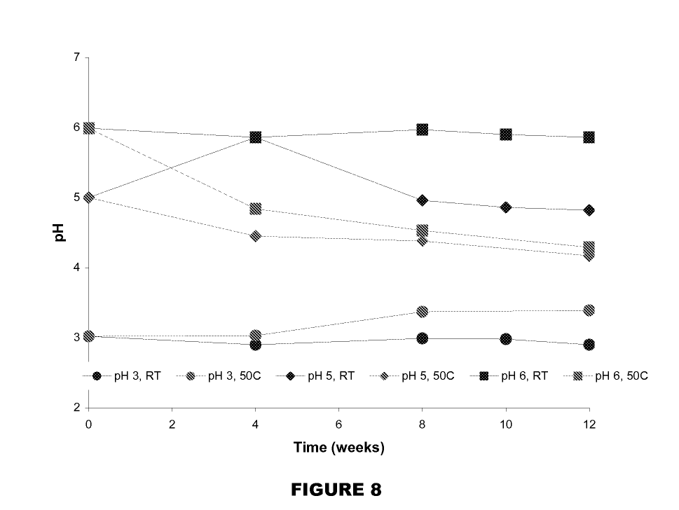

The pH values of all clindamycin analogs and BPO mixture samples after filtration are plotted versus time in FIGS. 4-8. The addition of CSOx analogs to the buffer solutions generally resulted in a pH increase by about 0.8-0.9 pH unit (FIGS. 4-6). Clindamycin phosphate addition to the buffers also yielded a pH increase but to a lesser extent (with the exception of pH 3 buffer, which remained essentially unchanged) (FIG. 7). No pH shift on the buffer solutions was observed from the addition of clindamycin HCl (FIG. 8). From FIGS. 4-6 it may be seen that a pH of about 4 seems to provide good stability of the clindamycin sulfoxides and clindamycin sulfone when mixed with benzoyl peroxide.

Compound Synthesis

The clindamycin sulfoxide forms CSO A and CSO B may be made by a non-stereospecific method, and then separated by an appropriate method, such as chromatography.

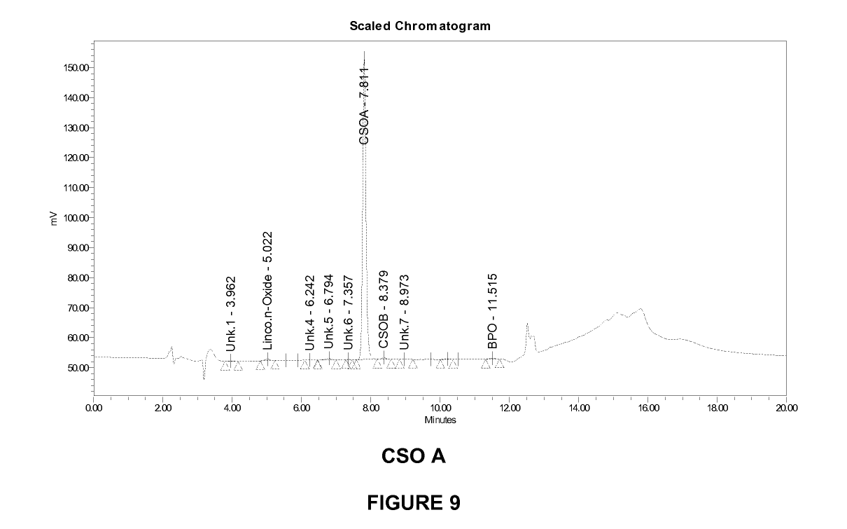

A stereospecific method of synthesizing CSO A is set forth in Scheme 1 below. The oxidation of clindamycin hydrochloride with iodobenzene dichloride was found to preferentially yield CSO A (MW=440.98). One test of this synthesis method yielded CSO A with greater than about 94% purity, as measured by HPLC. A scaled chromatogram of CSO A produced by this process is presented in FIG. 9.

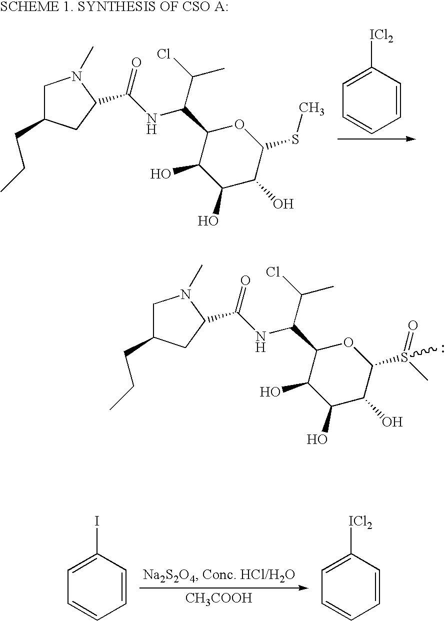

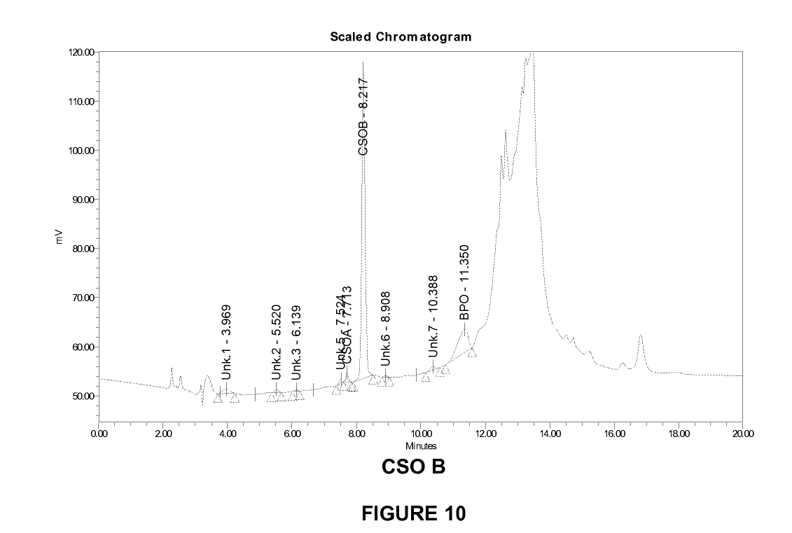

A stereospecific method of synthesizing CSO B is set forth in Scheme 2 below. The oxidation of clindamycin hydrochloride with sodium perborate (NaBO₃) at a pH between about 8 and 9 was found to preferentially yield CSO B (MW=440.98). One test of this synthesis method yielded CSO B with about 93% purity by HPLC. A scaled chromatogram of CSO B produced by this process is presented in FIG. 10.

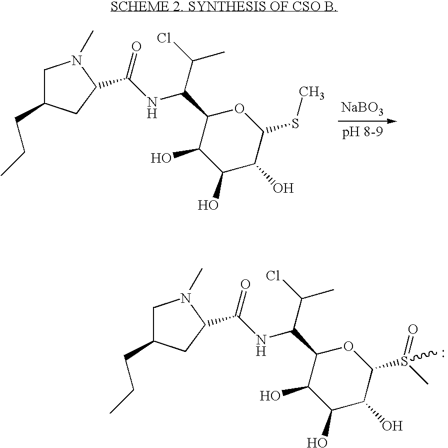

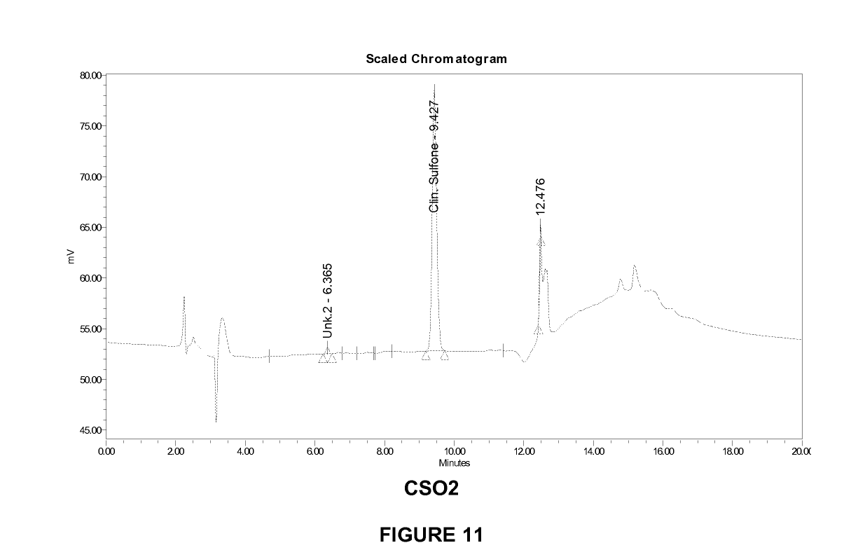

Clindamycin sulfone (CSO2) can be prepared directly by the oxidation of clindamycin HCl with hydrogen peroxide (H₂O₂), catalyzed by sodium tungstate (NaWO₄) at a pH of less than about 1, as shown in Scheme 3 below. In one test, this oxidation method yielded clindamycin sulfone (MW=456.98) with about 95% purity by HPLC. A scaled chromatogram of CSO2 produced by this process is presented in FIG. 11.

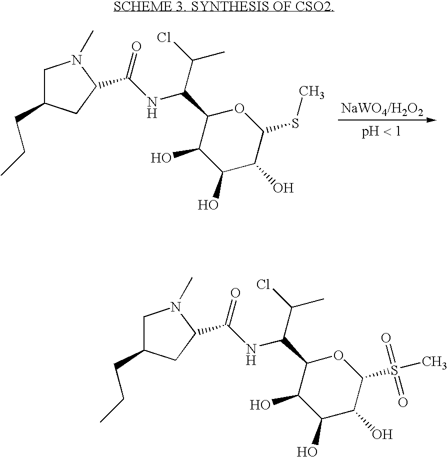

We claim:

1. A composition for the topical treatment of a dermatological condition comprising a sulfoxide or sulfone derivative of an antibiotic of the lincomycin family or a pharmaceutically acceptable salt or ester thereof.

2. The composition of claim 1 wherein the antibiotic of the lincomycin family is clindamycin or lincomycin, or a pharmaceutically acceptable salt or ester thereof.

3. The composition of claim 2 wherein the sulfoxide or sulfone derivative is a compound of the following formula I(a): 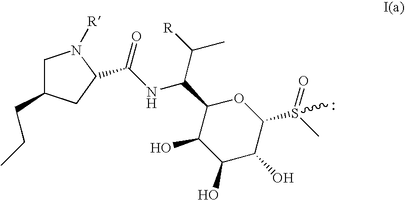
wherein R is hydroxy or halogen, and R′ is hydrogen or methyl, or a pharmaceutically acceptable salt or ester thereof.

4. The composition of claim 3 wherein the sulfoxide or sulfone derivative is a clindamycin sulfoxide of the following formula III, or a pharmaceutically acceptable salt or ester thereof:

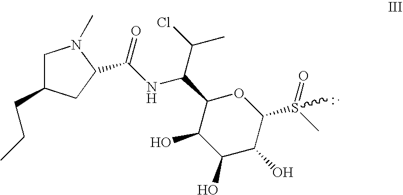

5. The composition of claim 4 wherein the sulfoxide or sulfone derivative is one or both stereoisomeric forms of clindamycin sulfoxide selected from the following formulas III(a) or III(b), or a pharmaceutically acceptable salt or ester thereof:

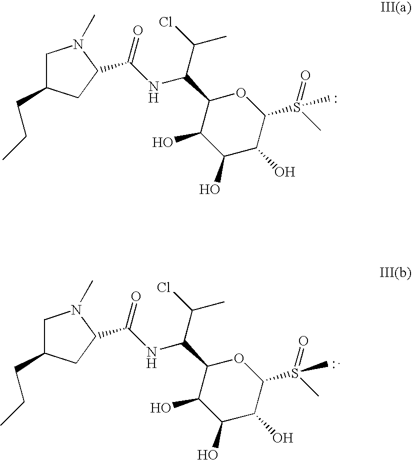

6. The composition of claim 2 wherein the sulfoxide or sulfone derivative is a clindamycin-2-phosphate sulfoxide of the following formula V, or a pharmaceutically acceptable salt or ester thereof:

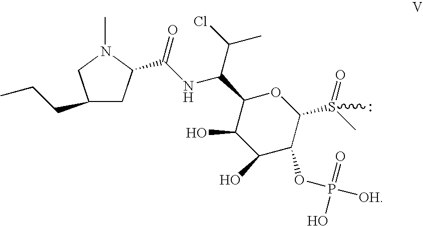

7. The composition of claim 6 wherein the sulfoxide or sulfone derivative is one or both stereoisomeric forms of clindamycin-2-phosphate sulfoxide selected from the following formulas V(a) or V(b), or a pharmaceutically acceptable salt or ester thereof:

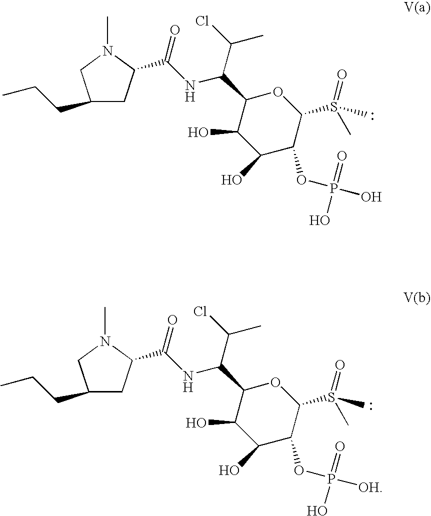

8. The composition of claim 2 wherein the sulfoxide or sulfone derivative is a compound of the following formula VI, or a pharmaceutically acceptable salt or ester thereof: 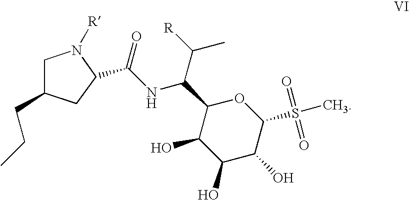
wherein R is hydroxy or halogen, and R′ is hydrogen or methyl, and pharmaceutically acceptable salts and esters thereof.

9. The composition of claim 8 wherein the sulfoxide or sulfone derivative is a clindamycin sulfone of the following formula VI(a), or a pharmaceutically acceptable salt or ester thereof:

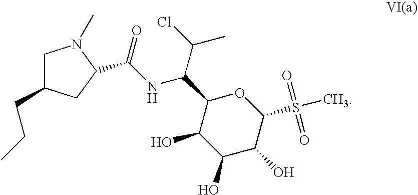

10. The composition of claim 1, further comprising a peroxide.

11. The composition of claim 10 wherein the peroxide is benzoyl peroxide.

12. The composition of claim 10 wherein the sulfoxide or sulfone derivative of the antibiotic is present in said composition in an amount of about 0.01 to about 10 weight percent of said composition and the peroxide is present in said composition in an amount of about 0.1 to about 30 weight percent of said composition.

13. A method for treating a dermatological condition comprising topically administering to a patient afflicted with such dermatological condition an effective amount of the composition of claim 1.

14. The method of claim 13 wherein said dermatological condition is acne.

15. The method of claim 13 wherein said dermatological condition is rosacea.

16. A pharmaceutical kit for the topical treatment of a dermatological condition comprising a first set and a second set of components, wherein said first set of components comprises the composition of claim 1, and said second set of components comprises a peroxide.

17. An antibiotic compound selected from the group consisting of 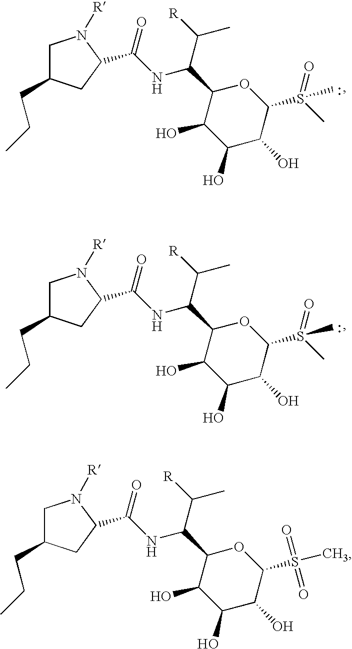
wherein R is hydroxy or halogen, and R′ is hydrogen or methyl, and the pharmaceutically acceptable salts and esters thereof.

18. The antibiotic compound of claim 17 selected from the group consisting of 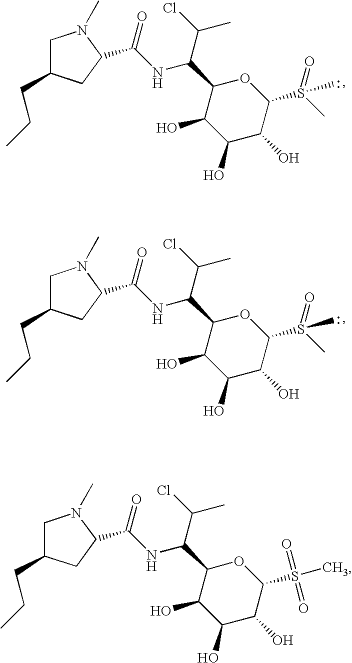
and the pharmaceutically acceptable salts and esters thereof.
